(19) United States
(12) Patent Application Publication
(10) Pub. No.: US 2010/0216256 A1
(43) Pub. Date: Aug. 26, 2010

(54) NANOBELT-BASED SENSORS AND DETECTION METHODS

(75) Inventors: Yi Cheng, Beltsville, MD (US); P. Bryant Chase, Tallahassee, FL (US); Nancy Meyer, Tallahassee, FL (US); Peng Xiong, Tallahassee, FL (US)

Correspondence Address: SUTHERLAND ASBILL & BRENNAN LLP, 999 PEACHTREE STREET, N.E., ATLANTA, GA, 30309, US

(73) Assignee: FLORIDA STATE UNIVERSITY RESEARCH FOUNDATION, Tallahassee, FL (US)

(21) Appl. No.: 12/707,284

(22) Filed: Feb. 17, 2010

Related U.S. Application Data
(60) Provisional application No. 61/153,203, filed on Feb. 17, 2009.

(51) Int. Cl. G01N 33/551; G01N 27/26; B29C 65/00

(52) U.S. Cl. 436/524; 436/86; 204/403.01; 156/60; 977/700

(57) ABSTRACT

A biosensor is provided which includes a substrate, a source electrode on the substrate, a drain electrode on the substrate, and at least one functionalized nanobelt on a surface of the substrate between the source electrode and the drain electrode. Methods for sensing a biological or chemical analyte using the sensor is also provided.

### CROSS-REFERENCE TO RELATED APPLICATIONS

This application claims benefit of U.S. Provisional Application No. 61/153,203, filed Feb. 17, 2009, which is incorporated herein by reference.

### BACKGROUND OF THE INVENTION

This invention relates generally to biosensors and detection methods, for example for use in applications such as medical diagnostics. In particular, the invention relates to nanoscale biological sensors.

Detection of bio-molecular substances such as virus, protein and DNA materials has long been a focus of research in biological and medical sciences. Conventionally, large complex equipment is used for such detection and analysis in a laboratory environment. Moreover, the detection speed of current biological detection methods is slow and typically limited by the diffusion speed of the molecules, which may result in the delay of the diagnostics and lack of timely medical treatment for some acute diseases. A need therefore exists for fast, light-weight, and portable biological sensors for various applications, including uses in medical emergencies such as cardiac arrest, in biological warfare defense, and in spacecrafts.

The history of solid-state biosensors can be traced back to 1960s. In the early literature, biosensors were categorized into enzyme electrodes, ion-selective electrodes, or biocatalytic membrane electrodes. In its modern context, a biosensor incorporates a biological element such as an enzyme, antibody, nucleic acid, microorganism, or cell. A biosensor is a compact analytical device incorporating a biological or biologically-derived sensing element either integrated within or associated with a physicochemical transducer. The usual aim of a biosensor is to produce either discrete or continuous physical (electronic, magnetic, mechanical, or optical) signals that are proportional to a single analyte or a related group of analytes. More generally, according to the International Union of Pure and Applied Chemistry recommendations in 1999, “a biosensor is a self-contained integrated receptor-transducer device, which is capable of providing selective quantitative or semi-quantitative analytical information using a biological recognition element.” A biosensor typically consists of three components: the detector, which recognizes the biological stimulus; the transducer, which converts the stimulus to a useful, measurable, output; and the output system, which generates amplification, display, etc., in an appropriate format.

Different nanotechnology-based approaches for biological detection, including electrical, mechanical, optical, and magnetic methods, have been reported. The reported results indicate that nanoscale biological sensors have the potential to overcome the limitations of previous detection technologies and redefine the boundaries of biological detection limits. Thus, there is interest in nanoelectronic devices that can detect the concentrations of biomolecules in real time for use as medical diagnostics (i.e., nanomedicine). A parallel line of research seeks to create nanoelectronic devices that can interact with single cells for use in basic biological research.

The selectivity of a biosensor for a specific target analyte may be impacted by the specificity and/or the presence of other, potentially interfering, species. Achieving an intrinsic signal and high selectivity, while at the same time avoiding a false signal and minimizing noise, remains a challenge for practical nanoscale biological sensor development. It would be desirable to provide a portable, efficient, reliable nanoscale biosensor. It would also be desirable to provide an integrated system combining miniaturization, low cost, and real-time measurements.

Statistical data from the American Heart Association reveals that cardiovascular diseases remain some of America's top fatal diseases. Accordingly, there is a need for rapid and accurate detection of several protein biomarkers (e.g., creatine kinase (CK), cardiac troponin T (TnT), and cardiac troponin I (TnI)) in the serum of patients as a tool for early evaluation of a heart disorder (e.g., after cardiac arrest). Of particular interest are cardiac protein biomarkers in cardiac troponin (cTn), which are released from damaged cells of heart muscles into the bloodstream upon trauma to the heart. Troponin is a complex of three regulatory proteins, TnC, TnI, and TnT, that plays a role in muscle contraction in skeletal and cardiac muscle. Cardiac troponin in cardiac muscle, specifically cardiac troponin I (cTnI), may be considered a promising marker. In the case of myocardial infarction (heart attack), the detection and analysis of these marker levels can be used by clinicians during the initial diagnosis and assessment of a patient's cardiac risk and for the timely selection of appropriate treatments.

A widely used laboratory technique for quantitatively determining these marker levels is enzyme-linked immunosorbent assay (ELISA), which has drawbacks in terms of cost, time, and ease of use.

It therefore would be desirable to provide improved and cost effective biological sensing devices and methods, particularly at the nanoscale level. It would also be desirable to provide biological sensors with high selectivity, particularly at the nanoscale level.

### SUMMARY OF THE INVENTION

A biosensor is provided. In one aspect, the biosensor comprises a substrate, a source electrode on the substrate, a drain electrode on the substrate, and at least one functionalized nanobelt on a surface of the substrate between the source electrode and the drain electrode. The functionalized nanobelt has a chemically functionalized surface linked to one or more detector molecules for binding with a biological analyte to be detected such that an electric field gating effect is generated by binding of the analyte to the one or more detector molecules linked to the nanobelt surface.

In certain embodiments, the substrate comprises Si/Si₃N₄. In some embodiments, the functionalized nanobelt comprises an oxide. In other embodiments, the functionalized nanobelt is prefunctionalized.

In some embodiments, the functionalized nanobelt is biotinylated. In one embodiment, the chemically functionalized surface is functionalized with (3-aminopropyl)triethoxysilane and biotin, and the one or more detector molecules comprises streptavidin linked to the (3-aminopropyl)triethoxysilane by the biotin.

In certain embodiments, the one or more detector molecules comprises an antibody linked to the chemically functionalized surface. In one embodiment, the antibody comprises a biotinylated antibody. In some embodiments, the biotinylated antibody comprises biotinylated anti-cTnI.

In some embodiments, at least one of the substrate, the source electrode, the and the drain electrode is passivated. In one embodiment, the sensor further comprises a back-gate.

In another aspect, a method of detecting a biological or chemical analyte in a fluid is provided. The method comprises contacting a fluid to be tested with a sensor. The sensor comprises at least one functionalized nanobelt. The functionalized nanobelt has a chemically functionalized surface linked to one or more detector molecules for binding with the biological or chemical analyte, wherein the binding of the biological or chemical analyte to the one or more detector molecules linked to the nanobelt surface provides a measurable electric field gating effect.

In yet another aspect, a method for making a sensor is provided. The method comprises functionalizing at least one nanobelt, linking one or more detector molecules to the functionalized nanobelt, and fabricating a field effect transistor comprising a substrate, a source electrode on the substrate, a drain electrode on the substrate, and the functionalized nanobelt on a surface of the substrate between the source electrode and the drain electrode.

### BRIEF DESCRIPTION OF THE DRAWINGS

FIG. 1 is a schematic illustrating one embodiment of a nanobelt-based field effect transistor (FET) sensor for sensing cTnI (cardiac troponin I).

FIG. 2 is a schematic illustrating one embodiment of a process for selective functionalization of a SnO₂ nanobelt FET device.

FIG. 3 is a schematic illustrating one embodiment of a method for in-solution functionalization of bundles of nanobelts and fluidic exchange with different functional molecules by repeated centrifuging and ultrasound agitation.

FIG. 4 is a schematic illustrating one embodiment of a FET fabrication procedure using prefunctionalized nanobelts.

FIG. 5 is a schematic showing the linking of APTES, biotin, and streptavidin to an oxide nanobelt in certain embodiments of the functionalized nanobelt-based FET sensors.

FIG. 6 shows an embodiment of a labeling reaction of a biotinylated antibody.

FIG. 7 is a micrograph comparing embodiments of fluorescently-tagged streptavidin on SiO₂ and Si₃N₄ substrates treated with APTES. The area encircled by the dotted lines on the top left is Si₃N₄ and the area encircled by the dotted lines on the bottom right is SiO₂.

FIG. 8 is a micrograph of an embodiment of a biotinylated nanobelt-based FET device exposed to a solution containing fluorescently-tagged streptavidin.

FIG. 9 is a schematic illustrating an embodiment of a nanobelt-based FET device setup and circuit for conductance measurement.

FIG. 10 is a graph of conductance over time, showing the real-time detection of hydrogen ions in sodium phosphate solutions having different pHs by an APTES modified SnO₂ nanobelt. The upper insert is a plot of time dependent conductance of the device as a function of the back-gate voltage. The bottom insert is a plot of conductance showing a linear dependence to pH.

FIG. 11 is a graph of conductance over time, showing conductance response of a SnO₂ nanobelt-based FET device before (showing no streptavidin recognition) and after APTES modification (showing reversible nonspecific binding) to a buffered streptavidin solution and a PBS buffer (pH=9).

FIG. 12 is a graph of conductance over time, showing conductance response of a biotinylated SnO₂ nanobelt-based FET to a PBS buffer and a buffered streptavidin solution (pH=7.2).

FIGS. 13A-B are graphs of conductance over time, showing conductance response of two biotinylated SnO₂ nanobelt FET devices to PBS buffer and PBS buffered streptavidin solution at pH 9 (FIG. 13A) and pH 4 (FIG. 13B). The inserts show the fluorescence images of the two devices after detection measurements.

FIG. 14 is a graph of conductance over time to show the electrical responses of open electrodes to PBS buffer and buffered streptavidin at pH 9.

FIG. 15 is a schematic illustration of another embodiment of a functionalized nanobelt-based FET device.

FIG. 16 is a graph of conductance over time, showing the response of a SnO, nanobelt FET functionalized with anti-cTnI to the cTnI and other control proteins.

FIG. 17 is a graph of conductance over time, showing the responses for an unmodified SnO₂ nanobelt FET device to cardiac troponin, tropomyosin, and BSA at different concentrations.

### DETAILED DESCRIPTION OF THE INVENTION

Functionalized nanobelt sensors and methods of manufacture thereof have been developed for use in biological sensing. In one aspect, the sensors include a field effect transistor comprising a substrate, a source electrode, a drain electrode, and a functionalized nanobelt. The functionalized nanobelt has a chemically functionalized surface linked to one or more detector molecules for binding with a biological analyte to be detected such that an electric field gating effect is generated by binding of the analyte to the one or more detector molecules linked to the nanobelt surface. Thus, the sensors described herein may also be referred to as nanobelt-based field effect transistor (FET) devices. In one embodiment, a solid-state biosensor includes a metal oxide nanobelt-based field effect transistor for electrochemical detection. In particular embodiments, sensors for electrically-based recognition of target proteins with biomedical relevance are provided. A method for detecting a biological or chemical analyte in a fluid is also provided. In one embodiment, the method comprises placing a field effect transistor in the fluid, wherein the field effect transistor comprises a functionalized nanobelt.

In preferred embodiments, the method uses a semiconducting nanobelt as the biosensing platform for transducing a molecule-protein or antibody-antigen interaction into an electrical signal. Through molecular assembly, chemical engineering, and biological functionalization of antibodies immobilized on the surface of the nanobelt, small amounts of protein markers in a small volume of solution (e.g., serum) may be detected. Due to the specific nature of the biological interaction between protein markers (i.e., antigens) and antibodies, an electrical field effect may be generated by the charges of the protein coupled onto the nanobelt so that the specific protein recognition (i.e., biological signal) may be converted qualitatively and quantitatively into a measurable electrical signal.

Among the three components of a biosensor, the biological detector or receptor confers to the biosensor its specificity. Thus, surface biochemical functionalization of the nanobelt-based FETs provides receptors for target molecules to be sensed. Without being bound by a particular theory, the conductance of the FET channel changes upon binding of charged target molecules to the receptors linked to the nanobelt surface. The electrical conductance modification is through electric field gating rather than direct exchange of charge carriers. The surface functionalization of the nanobelts includes self-assembly of macromolecules, selective surface functionalization, and minimizing the non-specific molecular bindings. In some embodiments, streptavidin protein sensing and antibodies for biomedically biomarker detections are provided.

An advantage of the nanobelt-based FET sensor is its portability. For example, the FET devices may be able to facilitate quick, onsite diagnosis of one or more heart disorders. In addition, nanobelts are inexpensively made by simple, catalyst free processes. By reducing the sensor size to about the size of the individual bio-molecules that are to be sensed, single molecule detection sensitivity may be possible. Thus, semiconducting oxide nanobelts are advantageously used in the sensors because of their small size and their electrical conductivity being sensitive to the type and concentration of molecules adsorbed on their surface. Development of miniaturized devices that enable rapid detection and direct analysis of specific proteins could be useful for not only medical diagnostic, but also for the exploration of new bio-molecular drugs. Such miniaturization on nanoelectronics towards in vivo proteomic sensing should enable new approaches to health monitoring, surveillance, and defense technology.

### The Sensors

In certain embodiments, the sensor comprises a substrate, a source electrode, a drain electrode, a functionalized nanobelt.

In some embodiments, the substrate comprises Si/Si₃N₄. In another embodiment, the substrate comprises Si/SiO₂. In other embodiments, the substrate includes any suitable material such as SiO₂ or other materials that are substantially non-reactive to the analyte environment and that have a minimal effect on the conductance of the functionalized nanobelt.

The source electrode and drain electrode may be made of any suitable materials known in the art for these FET components. For example, the source electrode and the drain electrode may comprise chromium, gold, a combination thereof, or the like.

In some embodiments, the source electrode and/or drain electrode is covered by a cap layer. For instance, cap layers may cover the source and the drain electrode in embodiments where the FET device is used to detect an analyte in a liquid solution. In other embodiments, the FET device is used to detect an analyte in a gaseous phase and does not include cap layers. The cap layer (or “insulating protective layer”) prevents or reduces electrical signal coming from the conduction of a fluid sample being measured (e.g., an electrolyte solution) through the electrodes (i.e., passivates the electrodes). In one embodiment, the cap layer comprises a dielectric material such as magnetron sputtered SiO₂ or Si₃N₄.

The functionalized nanobelt comprises an oxide or another suitable semiconducting material. In some embodiments, the nanobelt comprises a metal oxide. In one embodiment, the nanobelt comprises tin oxide (SnO₂), zinc oxide (ZnO), indium oxide (In₂O₃), or a combination thereof. In other embodiment, the nanobelt comprises a semiconducting material having a native —OH group on its surface. In should be understood that in particular embodiments, the nanobelt may comprise any semiconducting material having a suitable conductivity (or resistivity) for use in a FET device.

As used herein, “nanobelt” refers to a quasi-one-dimensional (Q1D) nanostructure in the form of a belt or strap. In certain embodiments, the nanobelt comprises a single crystal. In one embodiment, the nanobelts have an average length of less than 100 μm. In certain embodiments, the nanobelts are made by a physical vapor deposition process (PVD) without the use of a catalyst. In some embodiments, the nanobelt has a substantially rectangular cross-section. In some embodiments, the cross-section nanobelt is on the order of tens of nanometers.

In particular embodiments, the functionalized nanobelt includes molecules that chemically functionalize or link to the surface of the nanobelts. Suitable molecules for linking to the nanobelts include 3-aminopropyltriethoxysilane (APTES), biotin, and streptavidin, or other organic molecules with similar functional end groups. APTES has an amine group (—NH2) as an active terminal end, which can covalent bind to other small molecules such as biotin.

Biotin, also known as vitamin H or vitamin B7, has the chemical formula C₁₀H₁₆N₂O₃S and is a water-soluble B-complex vitamin found in all cells. D-biotin, which is also called succinimidyl ester, is one type of biotin and has the chemical formula C₁₄H₁₉N₃O₅S. The process of covalently attaching a biotin tag to a molecule or surface is called biotinylation as known in the art. For example, succinimidyl ester covalently attaches to APTES at the amine group. Biotin binds tightly to the tetrameric protein avidin and streptavidin, with a dissociation constant Kd on the order of 10⁻¹⁴ mol/L, which is one of the strongest known protein-ligand interactions. Streptavidin, a bacterial homologous protein to avidin, is a tetrameric protein purified from the bacterium Streptomyces avidinii.

The chemically functionalized surface of the nanobelts may be linked to one or more detector molecules for binding to an analyte. By providing a chemically functionalized surface for linking a detector to the nanobelts, different detector molecules may be easily and/or interchangeably linked to the nanobelts. In certain embodiments, the detector molecules include streptavidin, an antibody, avidin, or a combination thereof.

Antibodies, also known as immunoglobulin (Ig), are immunoglobulin proteins secreted by B white blood cells, which are present in serum or other body fluids. Antibodies combine specifically with certain type of antigens. Although the general structure of all antibodies is very similar, a small region at the tip of the protein is extremely variable, allowing millions of antibodies with slightly different tip structures to exist. This region is known as the hypervariable region. Each of these variants can bind to a different target, known as an antigen, which is any foreign substance that elicits an immune response. This diversity of antibodies allows the immune system to recognize an equally wide diversity of antigens. The part of the antigen recognized by an antibody is called an epitope or antigenic determinant. These epitopes bind with their antibodies in a highly specific interaction, called an induced fit, which allows the antibodies to identify and bind their unique antigen in the midst of the millions of different molecules that make up an organism. The interactions between antigen and antibody involve non-covalent binding of an antigenic determinant (epitope) to the variable region (complementarity determining region, CDR) of both the heavy and light immunoglobulin chains. These interactions are analogous to those observed in enzyme-substrate interactions and they can be defined similarly. To describe the strength of the antigen-antibody interaction, the affinity constant (K) may be defined as:

$$Affinity K = \frac{[ Ab - Ag ]}{[ Ab ] \cdot [ Ag ]}$$

Generally, the greater the K, the stronger the affinity between antigen and antibody. These interactions are the result of complementarity in shapes, hydrophobic interactions, hydrogen bonds, and Van der Waals forces. The affinity constants for antigen-antibody interaction typically fall in the range of 10⁴ to 10¹² mol⁻¹. The affinity constant for cTnI and its monoclonal antibodies and fragment antibodies is about 10⁹ to 10¹⁰ mol⁻¹. With such a high binding affinity, a large and irreversible conductance change results from bait antibodies immobilized on the nanobelt FET device bindings to target cTnI proteins.

In some embodiments, the FET sensor further comprises a back-gate. In particular embodiments, the back-gate comprises dope silicon or any other suitable gate materials known in the art. In one embodiment, the back-gate is provided to increase the number of charge carriers in the nanobelt above its intrinsic charge carrier level.

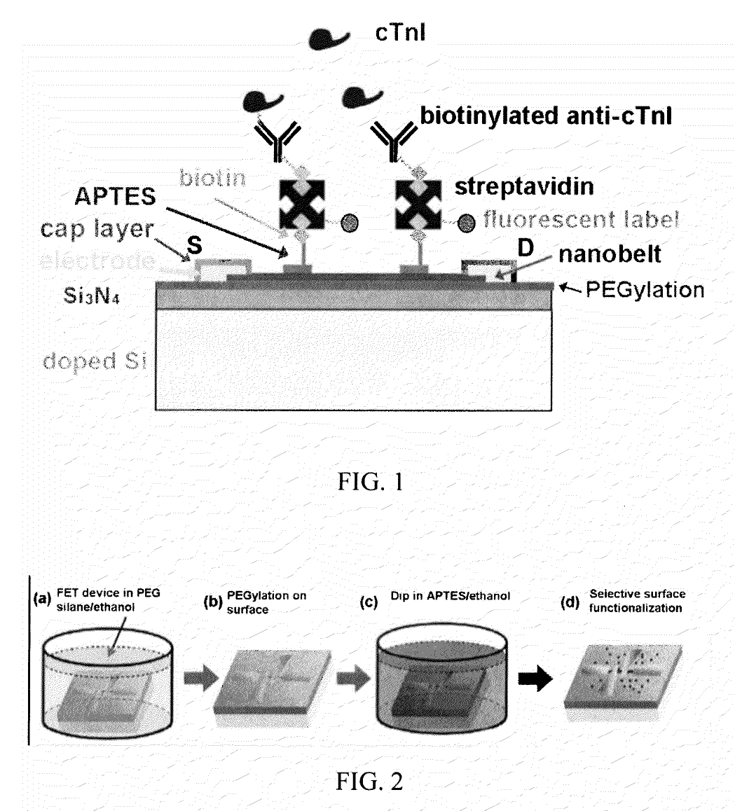

One embodiment of a nanobelt-based FET sensor for sensing cTnI (e.g., from a mammal such as a human or a rabbit) is shown schematically in FIG. 1. The FET sensor comprises a Si₃N₄ substrate, a doped silicon back-gate, a source electrode, a drain electrode, and a functionalized nanobelt linked to APTES, which is linked to streptavidin by biotin. A biotinylated antibody (anti-cTnI) is further linked to the streptavidin. The streptavidin is fluorescently labeled. Thus, the FET sensor is suitable for detecting cTnI that binds to the biotinylated antibody.

### Functionalization of the Nanobelts

Controlled nanoscale functionalization of the FET nanobelts of the sensors with biological substances may be used to achieve selective bio-detection. By functionalizing nanobelt surfaces with specific biological molecular units, biological sensors with high selectivity may be made. As used herein, the terms “functionalization” and “functionalize” refer to the creation of functional groups, cross-links, as well as various interconnections or junctions, on, in, and/or among the nanobelts.

In some embodiments, the nanobelt is functionalized after fabrication of the FET device. FIG. 2 illustrates one embodiment of a process for selective functionalization of a SnO₂ nanobelt FET device. First, the FET device is treated with polyethylene glycol (PEG) silane buffered in ethanol (e.g., a 1 vol. % PEG silane solution) to cover the FET surface uniformly with a monolayer of PEG silane. The PEG-silane binds to and passivates the SiO₂ substrate by forming an organic monolayer terminating with an alkoxy group (CH₃—O). Then, the FET is treated in APTES buffered in ethanol (e.g., a 2 vol. % APTES solution) for assembly of amino silane selectively onto the SnO₂ nanobelt surface by means of a longer treatment time. The amine group (NH₂—) on the SnO₂ nanobelt surface can covalently attach molecules like biotin (e.g., succinimidyl ester), which forms high infinity ligand-receptor binding to proteins like streptavidin tetramer.

Though the functionalization process shown in FIG. 2 provides a functionalized nanobelt-based FET, the long treatment times of the FET device with APTES in ethanol may be time-consuming and conducive to low specificity and a large amount of non-specific functionalization on the background substrate SiO₂. This result may be due to the competing binding on the hydroxyl enriched oxide surfaces (both SiO₂ and SnO₂) by APTES and PEG silane since they have the same silane end, or it could be due to the APTES monolayer replacing the alkoxy group (—OCH₃), which originally binds to the carbon chain in PEG silane.

In certain embodiments, selective nanobelt surface functionalization is achieved with higher specificity of the silanization on the SnO₂ surface as well as passivation on the background substrate. For instance, in embodiments where the substrate comprises Si/Si₃N₄, the non-specific binding of APTES on the substrate when functionalizing a tin dioxide nanobelt is reduced. Considering the nanoscale dimensions of the active sensing area of the nanobelt FETs, specific and/or nonspecific binding competition on the underlying dielectric SiO₂ layer will reduce the sensitivity. Due to the lower density of hydroxyl groups (—OH) on the surface, the Si₃N₄ substrate barely reacts with APTES and results in reduced APTES binding on the substrate.

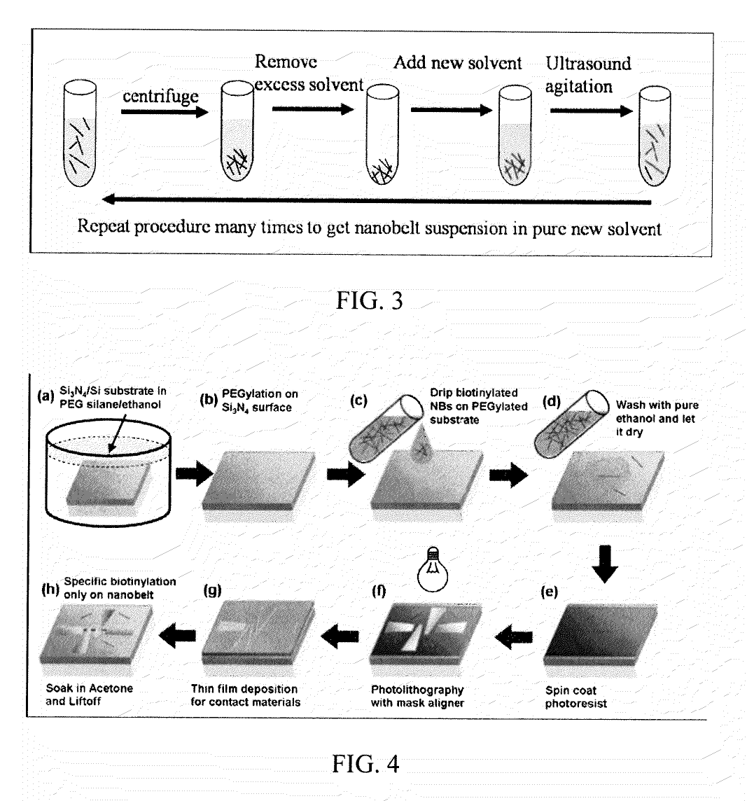

In other embodiments, the nanobelt may be functionalized without also functionalizing the other portions of the FET. In one embodiment, the nanobelts are functionalized before the device fabrication. A schematic of one embodiment of a method for in-solution functionalization of bundles of nanobelts and fluidic exchange with different functional molecules by repeated centrifuging and ultrasound agitation is illustrated FIG. 3. Bundles of nanobelts are sonicated in a suspension of an APTES solution buffered with ethanol to deposit a APTES layer onto the nanobelts surface (e.g., sonication for 10 minutes and then the nanobelts are maintained in the solution for 6 hours). By using repeated centrifuging, removing excess solution, adding new solution, and ultrasound agitating the solution, the APTES solution is removed and replaced with a N,N-Dimethylformamide (DMF) buffered D-biotin solution after the nanobelts are rinsed with ethanol. The bundles of nanobelts are kept in D-biotin solution for to allow the APTES on the surface of the nanobelts to react with the biotin (e.g., for 6 hours). Then, the nanobelts are dispersed in ethanol using the steps of centrifuging, removing excess solution, adding new solution, and ultrasound agitating of the solution. Lastly, the attachment of D-biotin molecules occurs through covalent bonding to the primary NH₂ group of the APTES on the oxide nanobelt surface. By using this method, the nanobelts are functionalized before being dispersed on a FET substrate.

Thus, in some embodiments, a Si₃N₄ substrate and selective pre-functionalization of the nanobelts, functionalization of the FET background (e.g., substrate) is minimized or reduced and the functionalization and molecular bindings are maintained on the nanobelt surface.

After the prefunctionalization (e.g., biotinylation) of the nanobelts, the nanobelts may be used to form FET devices. In one embodiment, illustrated schematically in FIG. 4, the FET device fabrication procedure includes the following steps: (1) biotinylated nanobelts are deposited onto a PEG-silane treated Si₃N₄ substrate by dripping an ethanol solution which contains biotinylated nanobelts onto the substrate; and (2) photolithography and thermal evaporation of Cr/Au are used to define source and drain electrodes to provide low resistance Ohmic contacts to the nanobelt. For in-solution protein sensing, the metal electrodes may be passivated. For example, the electrodes may be passivated with 80 nm of SiO₂ deposited by magnetron sputtering.

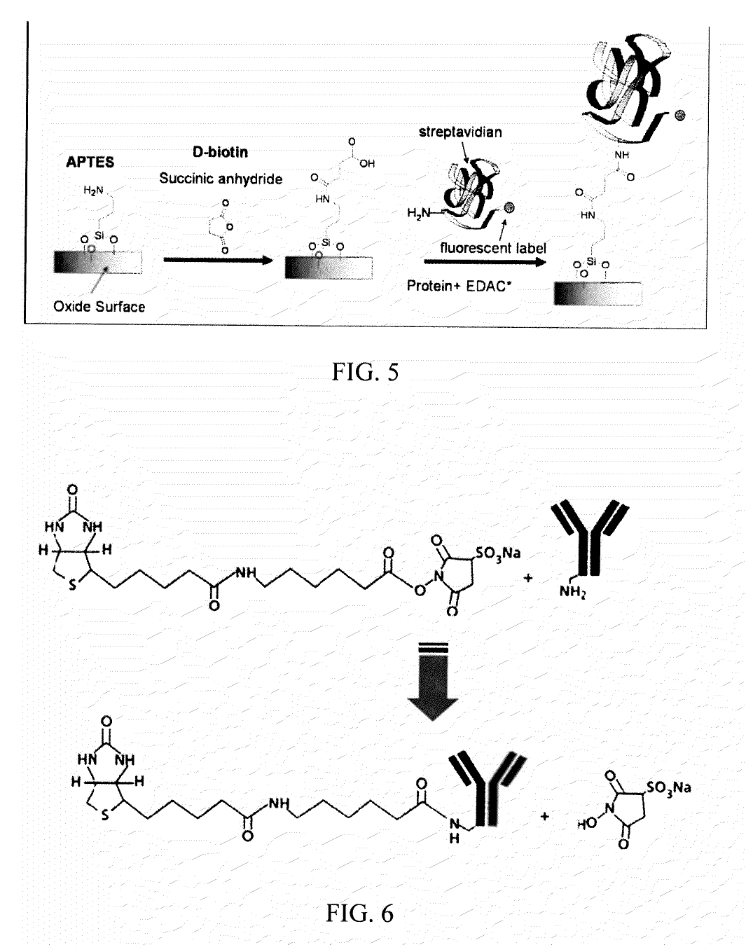

In one embodiment, a FET sensor is made by immobilization of the biotinylated antibody on the nanobelt surface using the following steps: A Si₃N₄ substrate is PEGylated by 1 vol. % PEG silane in ethanol for six hours in order to minimize non-specific bindings. Bundles of tin oxide nanobelts are surface-functionalized in a solution with 2 vol. % APTES in ethanol and D-biotin in N,N-Dimethylformamide (DMF) (10 mg/ml). To assemble specific antibodies for detecting the protein marker cTnI, the FET device is exposed to streptavidin in a phosphate buffered saline (PBS) buffer (10 μg/ml in 10 mM PBS) where streptavidins are linked on the biotinylated nanobelt surface. A schematic illustration of this linking is shown in FIG. 5. Since each streptavidin has four binding sites for biotin, subsequent introduction of biotinylated antibody (e.g., anti-IgG, such as from a rabbit) in PBS buffer (0.38 mg/ml) to the device leads to the linking of the antibody owing to the high affinity streptavidin-biotin binding.

In certain embodiments, an immunoprobe biotinylation kit (e.g., from Sigma-Aldrich BK-101) is used to label an antibody by conjugation of an amino group on the antibody to BAC-SulfoNHS (biotinamidehexanoic acid 3-sulfo-N-hydroxysuccinamide ester). BAC-SulfoNHS has two tetrahydrothiophene rings (a heterocyclic organic compound consisting of a five-membered ring containing four carbon atoms and a sulfur atom) and one of the rings is fused with a tetrahydroimidizalone ring. The amine group on an antibody replaces one of the tetrahydrothiophene rings and covalently attaches to the adjacent carbon, forming the biotinylated antibody. The labeling reaction (shown in FIG. 6) may proceed at room temperature for 30 min (or at 4° C. for 2 hrs).

### Sensing Applications for the Sensors

In certain embodiments, the nanobelt sensors may be used for real-time electrical protein sensing in an aqueous environment.

In some embodiments, biotinylated nanobelt FET devices are used for the electrical detection of streptavidin. The detection mechanism of streptavidin by the nanobelt FET is based on a surface gating effect. The conductance of the FET channel, the oxide nanobelt, is constant under the flow of certain pure buffer solutions, as detailed below. When a buffer solution containing streptavidin flows through the biotinylated device, the binding of charged streptavidin onto the biotinylated nanobelt surface creates an accumulation of surface charges. The electrical field caused by these charges therefore changes the distribution and concentration of charge carriers (e.g., electrons) in the semiconducting channel (i.e, the nanobelt) and changes the FET channel conductance. By monitoring the FET channel conductance change, the occurrence of specific biotin-streptavidin bindings is detected. Through the magnitude of the electrical signal (conductance change), the concentration of the streptavidin solution can be determined. The recognition of target protein requires reliable FET device performance, uniform coverage of nanobelt surface biotinylation, stability of the fluidic flow, proper function of the protein, appropriate pH of the buffer solution, and consistency between the electrical signals and the charge polarity of the charged proteins.

In particular embodiments, the nanobelt FET sensing platform is used for the detection of biomedically relevant biomolecules such as HcTcI.

Debye Screening Length

The nanobelt FET devices sense the presence of bound species by their intrinsic charge, with the advantage of enhanced sensitivity due to the nanoscale channel confinement. By assembling bait receptors on the nanobelt FET channel surface, the binding of specific ligands or proteins modifies the electric field surrounding the device, enabling direct electronic detection. As such, the effect of molecular charge screening by dissolved solution counterions (i.e., Debye screening) on the sensor response also may be considered in designing protocols for label-free sensing using oxide nanobelt FETs. The charge of solution-based molecules and macromolecules is screened by dissolved solution counterions (e.g., a negative species will surround positively charged ions due to electrostatic interactions). On a certain length scale, termed the Debye length (λ_{D}), the number of net positive charges approaches the number of negative charges on a protein. The result is a screening effect such that the electrostatic potential arising from charges on the protein decays exponentially toward zero with distance. For aqueous solutions at room temperature, this length is given by:

$${λ}_{D} = \frac{1}{\sqrt{4 π {l}_{B} {\sum }_{i}^{ } {ρ}_{i} {z}_{i}^{2}}}$$

where l_{B} is the Bjerrum length (=0.7 nm), Σ is the sum over all ion species, and ρᵢ and zᵢ are the density and valence, respectively, of ion species i.

Thus, the Debye length may selected for a particular nanobelt FET device. In some embodiments, the target proteins and the device surface are separated by 2-12 nm (e.g., contributions from APTES, biotins, and antibodies). The Debye length decreases with the increase of buffer ionic strengths. For example, 0.01×PBS buffer yields a Debye length of 7.3 nm, 0.1×PBS buffer yields a Debye length of 2.3 nm; and 1×PBS buffer yields a Debye length of 0.7 nm (1×PBS contains about 150 mM positive alkaline ions). Thus, the more diluted the buffer is, the greater the electrical signal will be.

The SnO₂ nanobelt FET devices and surface functionalization techniques facilitate label-free, real time, in-solution immunodetection. These quasi-one dimensional nanostructure-based FETs and nanoscale devices modified with specific receptors present a sensor platform for a broad range of biological and chemical species. These nanosensor-based devices allow for rapid, on-site evaluation of markers in a blood sample or other fluid samples.

It should be understood that the described techniques may transferred to other similar disease related biomarker detections with minor changes of surface linker molecules. For instance, functionalizations of nanobelt sensors could be based on commercially available antibodies, or could be based on specific interactions between CK monomers to form multimers; cTnT binding to tropomyosin; cTnI binding to troponin C or to the complex of troponin C plus T; commercially available mAbs (Monoclonal Antibodies) which are specific for the MB-CK (Myoglobin creatine kinase) isoform.

Although the described devices detect a particular protein, a single device that contains an array of nanosensors differently functionalized so as to be specific to different markers of interest.

The devices and methods described above will be further understood with reference to the following non-limiting examples.

### Example 1

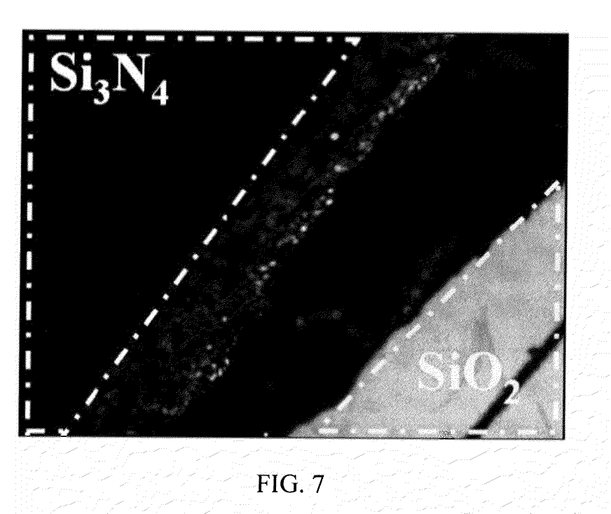

To compare SiO₂ and Si₃N₄ substrates treated with APTES, the substrates were reacted in fluorescently-tagged (Alexa-488) streptavidin solutions. First, the substrates were treated with 1 vol. % APTES solution buffered with ethanol for six hours. Then, the substrates were treated with D-biotin (5 mg) buffered with DMF (0.5 ml) for six hours. The substrates were rinsed with ethanol between each treatment to remove excess chemicals. A direct comparison of the two kinds of surfaces was performed using fluorescence microscopy and the result was shown in FIG. 7. The area encircled by the dotted lines on the top left is Si₃N₄ and the area encircled by the dotted lines on the bottom right is SiO₂. It was seen that the Si₃N₄ substrate is much less reactive to APTES than the SiO₂ substrate.

### Example 2

Nanobelts were prefunctionalized in this example. Physical vapor deposition (PVD) synthesized bundles of nanobelts were removed from a Al₂O₃ template and sonicated in a suspension in 1 vol. % APTES solution buffered with ethanol for six hours. Repeated centrifuge, removal of excess solution, addition of new solution, and ultrasound agitation was used to replace the 1 vol. % APTES solution with a N,N-Dimethylformamide (DMF) buffered D-biotin (5 mg in 0.5 ml) solution. The solution was sonicated for another six hours after rinse with ethanol, then the nanobelts were restocked and dispersed back in ethanol. D-biotin molecules were covalent bound to the primary NH₂ group of the APTES on the oxide nanobelt surface. For one sample, the nanobelt and the substrate were treated together in APTES and D-biotin by first dispersing bundles of nanobelts in ethanol. Then, the nanobelt solution was dripped onto the substrate and air dried. The substrate having the nanobelts on its surface was then treated with a 1 vol. % APTES solution buffered with ethanol for six hours followed by D-biotin (5 mg) buffered with DMF (0.5 ml) for another six hours. The substrate was rinsed with ethanol between each step in order to remove excess chemicals on the substrate surface. In the second sample, the nanobelt was prefunctionalized by APTES and D-biotin and then dispersed on the Si₃N₄. Both samples were then treated in solutions of fluorescently labeled streptavidin (0.01 mg/ml buffered in PBS buffer) to bind fluorescently tagged streptavidin to the biotin. The samples were both incubated for six hours, followed by a thorough rinse with PBS buffer and DI water. The fluorescent images of the two samples indicate the improved selectivity of the second functionalization process. By using the Si₃N₄ substrate and selective pre-functionalization the bindings on the background (i.e., substrate) were minimized and the desired functionalization and molecular bindings were maintained on the nanobelt surface.

Prefunctionalized (biotinylated) nanobelts were used to form FET devices. The FET device fabrication procedure is as follows: (1) a Si₃N₄ substrate was PEGylated by rinsing the substrate with ethanol before placing it in a PEG-silane solution (100 μl dissolved in 10 ml ethanol) for two hours; (2) biotinylated nanobelts were deposited onto the PEG-silane treated Si₃N₄ substrate by dripping the ethanol solution containing the biotinylated nanobelts onto the substrate; (3) to define the source and drain electrodes, the substrate was spin coated with AZ 520E photoresist at 5000 rpm for 40 s; (4) ultraviolet (UV) photolithography was carried out on a Karl Suss MJB 3 contact mask aligner with exposure intensity of 19 mW/mm²; and (5) thermal evaporation of Cr (10 nm)/Au (100 nm) at a vacuum level of 2×10⁷ torr was performed to metallize the source and drain electrodes to provide low resistance Ohmic contacts to the nanobelt; and (6) additional photolithography and magnetron sputtering of the SiO₂ (80 nm) as electrode cap layers, and lift-off were performed.

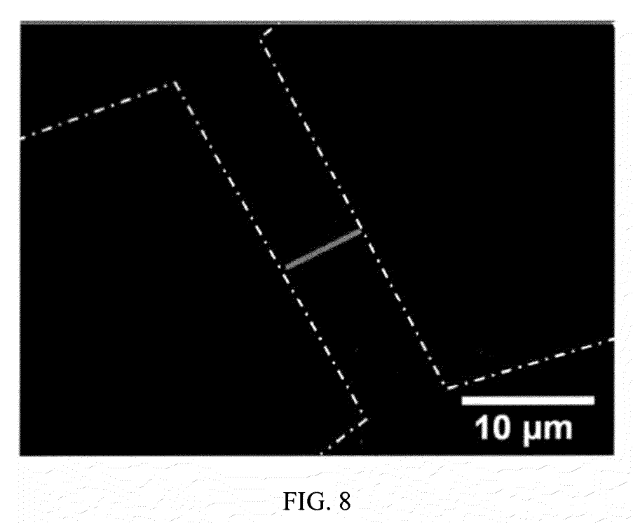

The prefunctionalization (the biotin monolayer) survived the vacuum deposition and lithography processes and remained functional. This was verified by exposing an as-made FET device to fluorescently labeled streptavidin and observing it using fluorescence microscopy. The resulting micrograph is shown in FIG. 8. The white dotted lines in FIG. 8 depict the edges of the SiO₂ capped electrodes. Bright fluorescence is confined exclusively to the SnO₂ nanobelt with uniform coverage of fluorescently tagged streptavidin in the center of the micrograph. The fluorescence from the PEG-silane passivated Si₃N₄ substrate is negligible. The image suggests that the functionalization process achieved a high density binding of streptavidin on the nanobelt channel, most likely through the biotin linkage and there was minimal non-specific binding on the substrate and capped electrodes.

### Example 3

In order to establish the specificity of a biosensing scheme, biosensors were fabricated to verify the biomolecular binding from multiple means (multi-modality) and to perform an assortment of control experiments to rule out the possibility that the signal originating from or was modified by the binding of other proteins.

To demonstrate the electrical detection of the biotin-streptavidin binding, in-solution protein sensing experiments were performed using microfluidics. A microfluidic channel was made from a gel-like polydimethylsiloxane (PDMS) liquid and a curing agent with a photolithographically defined master. The structure had two reservoirs connected by a channel (100 μm wide and 80 μm high), each with an inlet or outlet. The solidified transparent PDMS replica was placed on the nanobelt FET with the microfluidic channel covering the exposed active portion of the SnO₂ nanobelt and parts of the passivated source/drain electrodes. The solution flow was initiated via a syringe pump evacuating on the outlet. With a flow rate of about 30 μl/min, a small amount of analytes of just buffer and also buffer which contains streptavidin can be delivered to the active sensing area of the nanobelt FET.

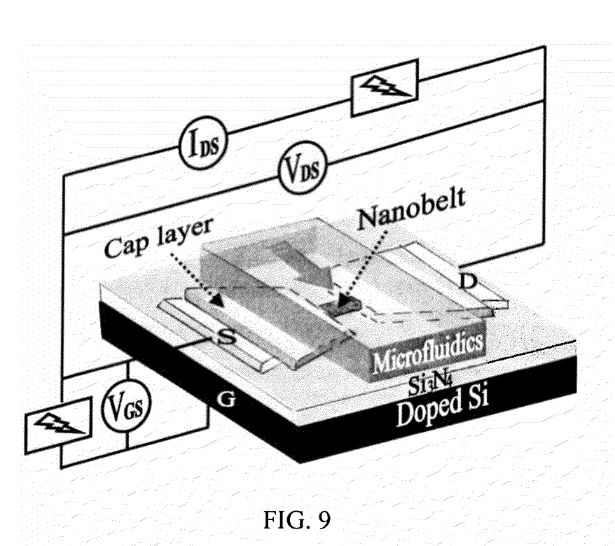

FIG. 9 shows a schematic illustration of an embodiment of a FET device having a Si₃N₄ substrate, source and drain electrodes covered with cap layers, a doped silicon back gate, and a functionalized nanobelt in use with a microfluidics system. The current applied may be from the source to the back gate and from the source to the drain may be supplied by a battery (denoted by the rectangle including a lighting bolt).

Fluorescence microscopy was used after electrical detection measurements to independently verify that the FET electrical signal was indeed due to the biotin-streptavidin binding. Streptavidin labeled with CdSe quantum dots was used with a 585 nm emission spectrum for the fluorescence microscopy. These quantum dots semiconductor nanocrystals, whose band gap was modified from the bulk value due to the quantum confinement, have advantages such as size- and composition-tunable emission from visible to infrared wavelengths, large absorption coefficients (absorption rates are 10-50 times faster than that of organic dyes at the same excitation photon flux) across a wide spectral range, high levels of brightness (10-20 times increased rate of light emission), and photostability (several thousand times more stable against photo bleaching than dye molecules). The surface modification process schematically depicted in FIG. 5 was performed with the quantum dot labeled streptavidin to a biotinylated nanobelt and the distribution of streptavidin coverage was studied under a fluorescence microscope. The biotinylation of the nanobelt was performed in the same manner as described for the second sample of Example 2. Based on the fluorescence microscopy, high selectivity was seen for the functionalization of the nanobelt.

### Example 4

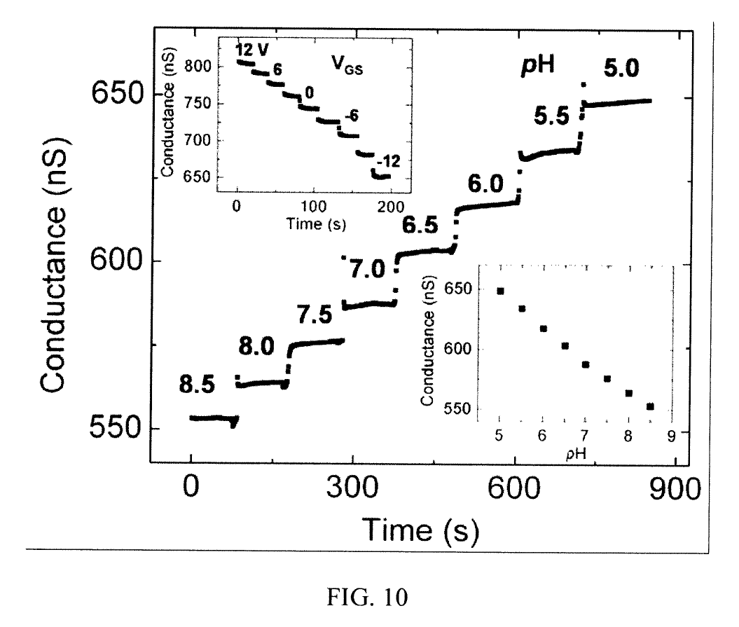

In this example, APTES functionalized devices, which have been characterized as a hydrogen ion sensors, exhibiting a linear conductance dependence on the buffer solution pH, were made in the same manner as described for the second sample of Example 2 (except without the biotinylation step). By flowing sodium phosphate buffers with various pH values across the devices, the hydroxyl end (—OH) and amine end (—NH2) on the surface of the functionalized oxide nanobelt were protonated and deprotonated. For an n-type semiconductor, as the acidity increases, the protonation on the surface acts as a positive gate which contributes carriers (electrons) in the underlying channel, and thus results in a conductance increase. As the acidity decreases, the deprotonation on the surface decreases the channel conductance due to the depletion of the carriers, similar to the effect of a negative gate. The response of a SnO₂ FET device to eight solutions with pH values varying from 8.0-5.0 is shown in FIG. 10. For comparison a time-dependent conductance of the same device as a function of back-gate voltage was also displayed in the top inset in FIG. 10, indicating that in the linear transport regime, the conductance of the device has a linear dependence on the pH value of the 10 mM sodium phosphate buffer solutions with a sensitivity of 28 nS/pH (bottom inset, FIG. 10).

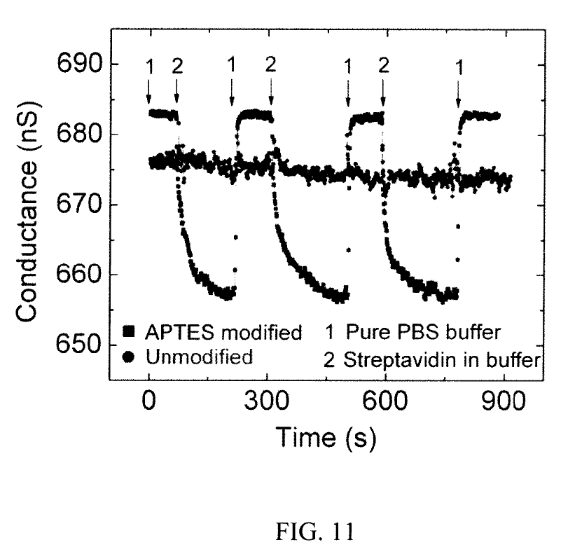

In order to estimate the degree of non-specific streptavidin binding (not through binding with biotin) on the SnO₂ surface and its effect on the electrical sensing measurement, a set of control experiments of protein detection were carried out with a device before and after the APTES modification. The result is shown in FIG. 11. PBS buffer and also streptavidin in buffer were separately delivered to a SnO₂ nanobelt FET device with and without APTES treatment. The bare SnO₂ device showed no conductance change upon the introduction of streptavidin in a PBS buffer (pH=9) from a PBS buffer at the same pH, indicating a low or negligible affinity of the streptavidin to bare SnO₂ surface. In contrast, for an APTES modified device, the introduction of the streptavidin solution did induce a conductance change. The binding of negatively charged streptavidins on an n-type semiconducting SnO₂ nanobelt induced a decrease of conductance due to the electrostatic gating effect. However, this type of streptavidin binding, due to the amine (—NH₂) functional group on the surface, was reversible. This was evidenced by the conductance exhibiting a full and repeatable recovery upon reintroduction of the PBS buffer. Upon switching the flow back and forth from buffer and streptavidin solution, the device exhibited consistent conductance increase and decrease corresponding to protein binding and unbinding.

### Example 5

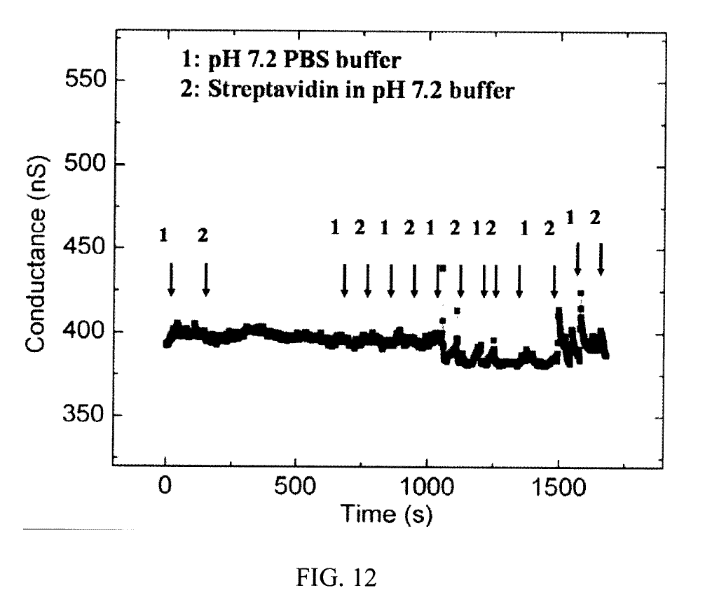

Real-time, label-free sensing of streptavidin was carried out by monitoring the electrical responses of a biotinylated SnO₂ nanobelt FET device to PBS buffer (10 mM) and Q-dot tagged streptavidin in PBS buffer. The biotylation of the SnO₂ nanobelt was in the same manner as described for the second sample of Example 2. The solutions were at a pH of 7.2. The PBS buffer did not induce any conductance change. The PBS buffered streptavidin solution also did not cause any significant conductance variation either (FIG. 12). This lack of conductance variance was due to the pH of 7.2 being close to the isoelectric point (pI) of streptavidin. The pI of streptavidin is 5-6 (see Table 1). The charged state of biomolecules in a solution depends on the solution pH. The amount and the polarity of the charge can vary with the pH value. The isoelectric point for a molecule is the pH value at which the molecule is neutral (uncharged) in the solution. The pH of 7.2 is close to the pI of streptavidin, which is the reason for the null result in FIG. 12. The conductance response of the nanobelt FET should vary systematically as the pH value of the streptavidin solution is varied, in particular, it should change sign across the isoelectric point.

TABLE 1
Estimated surface charge of streptavidin at different pH.

| pH | Charge |
|---|---|
| 3.00 | 14.1 |
| 3.50 | 12.8 |
| 4.00 | 10.4 |
| 4.50 | 6.8 |
| 5.00 | 3.6 |
| 5.50 | 1.8 |
| 6.00 | 0.8 |
| 6.50 | 0.1 |
| 7.00 | −0.6 |
| 7.50 | −1.1 |
| 8.00 | −1.6 |
| 8.50 | −2.2 |
| 9.00 | −3.2 |
| 9.50 | −5.3 |
| 10.00 | −9.0 |

* Direct molecular level measurements of the electrostatic properties of a protein surface, Proc. Natl. Acad. Sci. USA, (95), 1998

Thus, a higher absolute value of the difference between pI and solution pH provides a better sensing response. Thus, streptavidin sensing using the FET devices in solutions at pH of 9 and 4 was performed. These pH values were further from the pI at the two opposite sides, but safely avoided any protein denaturing. The results for solutions at pH=9 and 4 are shown in FIGS. 12A-B, respectively. The device exhibited a conductance decrease upon the introduction of a streptavidin solution (2 ng/ml) buffered with a pH=9 PBS solution. The FET channel conductance decreased by about 26.83%. The decrease is consistent with the negatively charged state of the streptavidin at this pH, which when binding on the biotinylated SnO₂ nanobelt surface acts as a negative gate and depletes carriers (electrons) in the channel and results in conductance decrease. In contrast, at a buffer pH below the pI (pH=4), the streptavidin solution induced a large increase in conductance (about 133%). The conductance is expected from the positively charged state of streptavidin at this pH, which acts as a positive gate and induces carriers (electrons) in the FET channel upon binding.

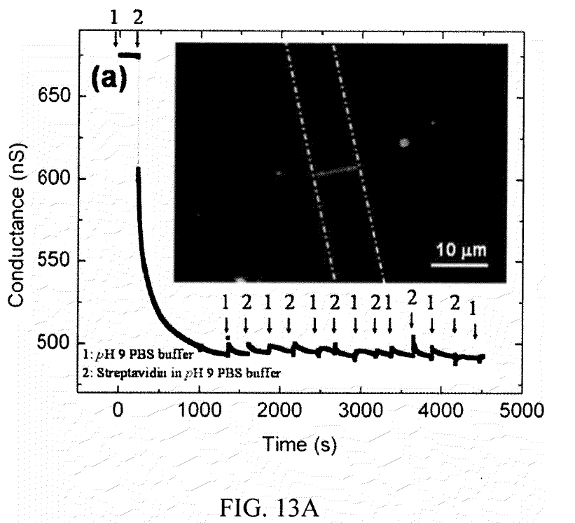

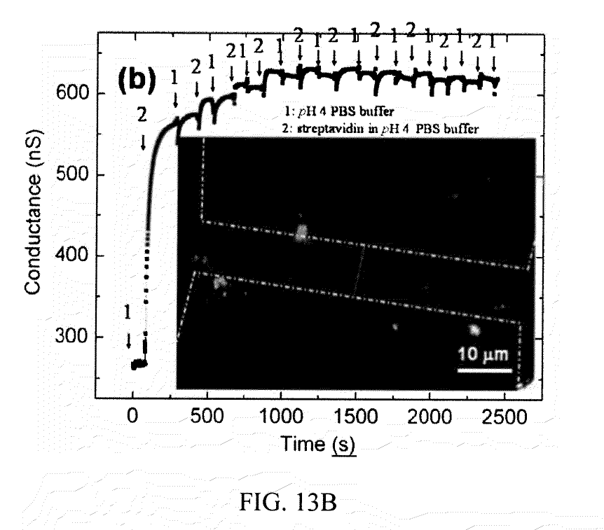

In both cases, the pure PBS buffer and buffered streptavidin solution were flowed back and forth many times after the initial introduction of the streptavidin solution. The sensor responses indicate that the bindings were predominantly irreversible. At the subsequent switching of the solution flows, the conductance fluctuated within a small range upon the switching, suggesting a small number of disassociated bindings. This is consistent with the high affinity but non-covalent binding (Ka=1015 M−1) of the biotin-streptavidin ligand-receptor system. In FIG. 13B, after the initial introduction of streptavidin, the pure buffer was flowed much earlier (t=300 s) than the time point in FIG. 13A. Consequently, when additional streptavidin solution was delivered again through the device, the conductance showed further increases but eventually saturated. The saturation paces for the two devices in FIGS. 12A-B are similar (about 1000 s). The reason for the long saturation time is most likely the small concentration of the streptavidin solution (2 ng/ml) and large surface area of the nanobelt (width about 640 nm and 420 nm in FIG. 13A-B respectively).

The bindings of the streptavidin were further confirmed by fluorescence microscopy performed right after the electrical sensing experiments. The images are shown in the insets of FIGS. 12A-B. The white dotted lines indicate the edges of the SiO₂ capped electrodes. The fluorescence from the Q-dots was confined exclusively to the nanobelts. Along with the pH dependence of the electrical detection results, this shows that the streptavidin binding is confined to the nanobelt and it was the streptavidin binding that causes the electrical conductance change.

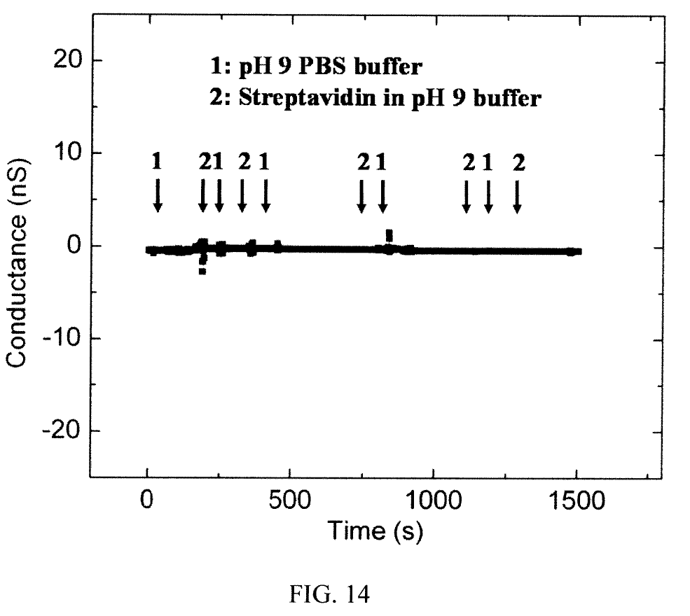

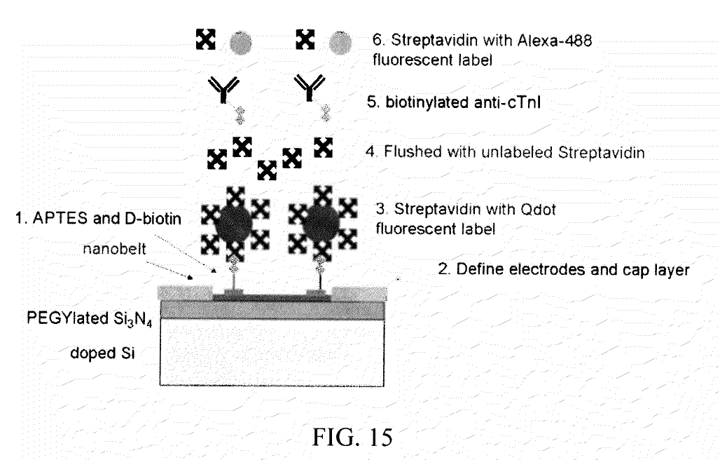

Although the Cr/Au electrodes were passivated with SiO₂, due to the high conductivity of the SnO₂ nanobelt (from the surface oxygen deficiency), it was possible that the electrical signals may contain contributions from ionic conduction through the electrolyte solution, as shown in the pH sensing experiments at high ionic concentrations. A control experiment (FIG. 14) was conducted in which the electrical responses of a device with open electrodes (no nanobelt connecting the electrodes) were monitored in response to solution flows. The conductance response was immeasurably small. Thus, the contribution to electrical signal from ionic conduction through electrolyte was negligible.

### Example 6

The retention of the functionality and specificity of the antibody after biotin labeling was confirmed by comparison of western blots of biotinylated and unbiotinylated antibody bindings to cardiac troponin complex at different concentrations. The western blots results also suggested concentrations of antibodies for effective binding to the corresponding troponin sub units.

A biotinylated antibody has one antibody site which has high affinity to the cTnI as well as two active sites on the biotin which can bind to streptavidin. Therefore, after the biotinylated antibodies bind with the streptavidins anchored on nanobelt surface, they still are capable of binding to free streptavidin with the available biotin sites. To verify the binding of biotinylated antibody onto the streptavidin immobilized on the oxide nanobelt, a two-color fluorescence scheme was used to confirm the binding of the biotinylated antibody to the nanobelt. First, the proper biotinylation of the nanobelt with Q-dot labeled streptavidin was confirmed; second, the existence of biotinylated antibody with Alexfluor 488 labeled streptavidin.

A schematic illustration of the FET and molecules used for the verification experiment are illustrated in FIG. 15. The experiment was carried out as follows: (1) bundles of nanobelts were functionalized with APTES and D-biotin in solution before the fabrication of the FET device; (2) a biotinylated nanobelt was placed on a PEGylated Si₃N₄ substrate, electrodes with a protective layer were deposited on the substrate, and a FET structure was formed; (3) the FET sample was treated with Q-dot fluorescent labeled streptavidin in a PBS buffer; (4) the FET sample was flushed with unlabeled streptavidin in PBS buffer and then just PBS buffer in order to saturate the unused biotin binding sites on the nanobelt; (5) antibodies reacted with BAC-SulfoNHS in PBS buffer and then just PBS buffer (biotinylated antibody) were introduced to the FET device with the streptavidin-coated nanobelt in order to immobilize the antibodies on the nanobelt; (6) the FET sample was flushed with Alexafluor-488 fluorescent labeled streptavidin in PBS buffer and then just PBS buffer to verify the proper assembly of the biotinylated antibodies. The sample was characterized under a fluorescence microscope after step (3) and step (6) using difference emission filters. A center wavelength matching emission filter was used to view the quantum dot labeled (emission spectrum peak near 655 nm) streptavidin and the Alexafluor-488 labeled (emission spectrum peak near 488 nm) streptavidin. The microscopy showed selective surface biotinylation of the nanobelt and uniform coverage with high specificity and contrast with the background substrate. The image demonstrated the presence of the biotinylated antibodies on the nanobelt surface and their uniform coverage along the channel.

### Example 7

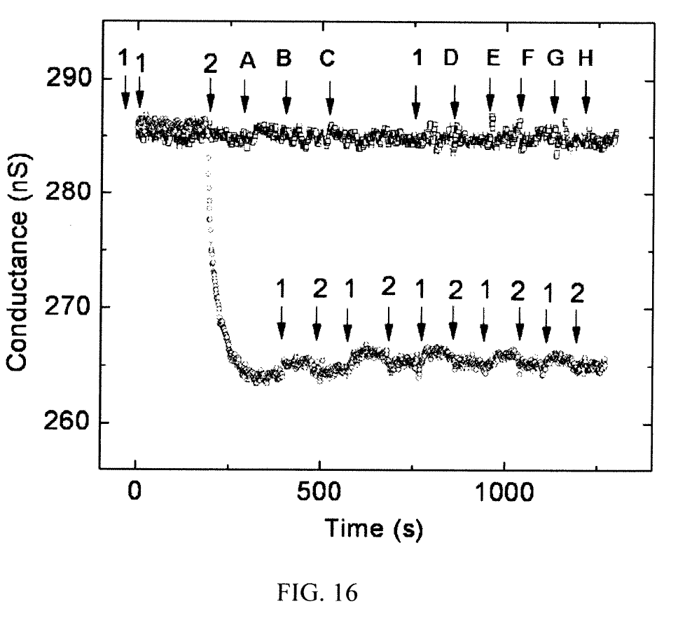

cTnI protein sensing and control experiments at diluted buffer concentrations were performed in order to establish that the sensor signal originates exclusively from the biomolecule of interest, while other similar molecules do not induce any sensor response. FIG. 16 shows the response of a SnO₂ nanobelt FET functionalized with anti-cTnI to the cTnI and several other control proteins. Because the anti-cTnI IgG is the specific antibody for cardiac troponin I, it should not bind with other proteins such as tropomyosin and bovine serum albumin (BSA). In order to confirm the specificity of the FET electrical sensing, before introducing the cTnI solution to the antibody modified nanobelt FET, varying concentrations of control protein tropomyosin and BSA were flowed to the device. The results are shown with the almost straight curve in FIG. 16. Number 1 represents the 10 mM PBS buffer in pH of 7.5. Letters A-C represent 5.75, 11.5 and 23 nM tropomyosin in 10 mM PBS buffer. Letters D-H represent 1.56, 6.25, 25, 100 and 400 nM BSA in 10 mM PBS buffer. Number 2 represents 25 nM cTn in 10 mM PBS buffer.

The absence of any conductance change in the signal demonstrated that the functionalized nanobelt FET device response was independent of the presence of the control proteins. The results were in good agreement with the expectation that anti-cTnI IgG has high binding affinity to its own antigen. On the other hand, when switching from 10 mM pure PBS buffer to 25 nM cTn in the same buffer to the same device, a conductance decrease was observed. Furthermore, a switch of the flow of PBS buffer and then cTn complex back and forth several times resulted in the device conductance showing minimal shifts (FIG. 16, downward sloping curve). The initial conductance decrease was due to the binding of negatively charged troponin complex on the antibody modified SnO₂ nanobelt (n-type semiconductor) surface. The polarity and the magnitude of surface charge of cardiac troponin complex are shown in Table 2. The negative charges on the SnO₂ nanobelt surface created an electrical field which gated the conducting FET channel by depleting the electron carriers inside. Thus the conductance of the SnO₂ nanobelt decreased. The binding affinity between the cTnI and its antibody is on the order of 10⁹-10¹⁰ mol⁻¹. Therefore, the minor conductance fluctuation after the initial drop was possibly owing to the small unbinding and rebinding of the antibodies and antigens on the nanobelt surface.

TABLE 2
Polarity and Magnitude of Surface Charge of Cardiac Troponin Complex.

| pH | Charge |
|---|---|
| 3.00 | 131.4 |
| 3.50 | 119.3 |
| 4.00 | 91.2 |
| 4.50 | 47.2 |
| 5.00 | 8.1 |
| 5.50 | −12.8 |
| 6.00 | −21.8 |
| 6.50 | −26.3 |
| 7.00 | −29.2 |
| 7.50 | −31.1 |
| 8.00 | −33.0 |
| 8.50 | −36.1 |
| 9.00 | −42.2 |
| 9.50 | −54.8 |
| 10.00 | −76.0 |

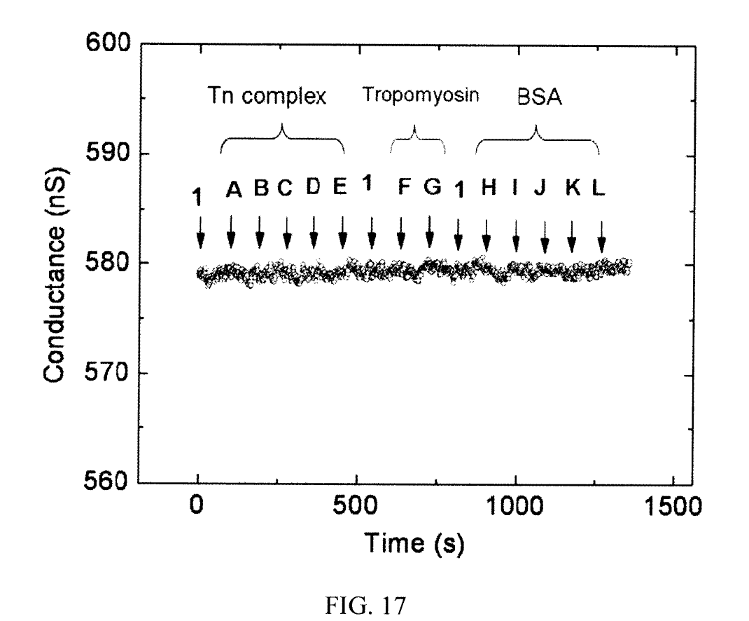

To evaluate the significance of the surface antibody functionalization and to compare the difference in signal from an unmodified SnO₂ nanobelt, a set of control experiments were carried out. A SnO₂ nanobelt FET device was fabricated without any surface functionalization by the following steps: bundles of nanobelts were put into ethanol and sonicated to form a suspension; the nanobelt solution was dripped onto a Si₃N₄ substrate; and a nanobelt was located and then the source and drain electrodes were patterned by photolithography, therma evaporation of Cr (10 nm)/Au (100 nm), and lift-off was performed. Additional photolithography, sputtering of SiO₂ (80 nm) as an electrode cap layer, and lift-off were performed. Three sets of electrolytes with three different kinds of proteins were brought in contact with the unmodified device. The result is plotted in FIG. 17. Letters A-E represent 1.56, 6.25, 25, 100, 400 nM cardiac troponin complex in 10 mN PBS buffer. Letters F-G represent 5.75 and 213 nM tropomyosin in 10 mM PBS buffer. Letters H-L represent 1.56, 6.25, 25, 100, and 400 nM BSA in 10 mM PBS buffer. Upon introducing cardiac troponin complex in buffer at the concentrations from 1.56 to 400 nM (FIG. 17, solution A-E), the unmodified device showed no significant conductance change. Similar results were obtained for tropomyosin (FIG. 17, solution F, G) and BSA (FIG. 17, solution H-L). The results suggest that the cardiac troponin sensing using SnO₂ nanobelt FET device is possible when the nanobelt surface is properly functionalized with the appropriate antibody. The hydroxyl groups on the native tin oxide surface show no affinity to any of the surface charged proteins.

The successful nanobelt FET devices fabrication and characterization, and their in-depth examination in fluid sensing and pH sensing demonstrated that these oxide nanobelts FET devices are promising for biosensing applications.

Modifications and variations of the methods and devices described herein will be obvious to those skilled in the art from the foregoing detailed description. Such modifications and variations are intended to come within the scope of the appended claims.

We claim:

1. A biosensor comprising:
a substrate,
a source electrode on the substrate,
a drain electrode on the substrate, and
at least one functionalized nanobelt on a surface of the substrate between the source electrode and the drain electrode,
wherein the functionalized nanobelt has a chemically functionalized surface linked to one or more detector molecules for binding with a biological analyte to be detected such that an electric field gating effect is generated by binding of the analyte to the one or more detector molecules linked to the nanobelt surface.

2. The biosensor of claim 1, wherein the substrate comprises Si/Si₃N₄.

3. The biosensor of claim 1, wherein the functionalized nanobelt comprises an oxide.

4. The biosensor of claim 1, wherein the functionalized nanobelt is prefunctionalized.

5. The biosensor of claim 1, wherein the chemically functionalized surface is functionalized with (3-aminopropyl)triethoxysilane and biotin, and wherein the one or more detector molecules comprises streptavidin linked to the (3-aminopropyl)triethoxysilane by the biotin.

6. The biosensor of claim 1, wherein the chemically functionalized surface is biotinylated.

7. The biosensor of claim 1, wherein the one or more detector molecules comprises an antibody linked to the chemically functionalized surface.

8. The biosensor of claim 7, wherein the antibody comprises a biotinylated antibody.

9. The biosensor of claim 8, wherein the biotinylated antibody comprises biotinylated anti-cTnI.

10. The biosensor of claim 1, wherein at least one of the substrate, the source electrode, and the drain electrode is passivated.

11. The biosensor of claim 1, further comprising a back-gate on the substrate.

12. A method of detecting a biological or chemical analyte in a fluid comprising:
contacting a fluid to be tested with a sensor, which comprises at least one functionalized nanobelt,
wherein the functionalized nanobelt has a chemically functionalized surface linked to one or more detector molecules for binding with the biological or chemical analyte, wherein the binding of the biological or chemical analyte to the one or more detector molecules linked to the nanobelt surface provides a measurable electric field gating effect.

13. The method of claim 12, wherein the method is used to detect cardiac troponin.

14. The method of claim 12, wherein the substrate comprises Si/Si₃N₄.

15. The method of claim 12, wherein the functionalized nanobelt comprises an oxide.

16. The method of claim 12, wherein the functionalized nanobelt is prefunctionalized.

17. The method of claim 12, wherein the chemically functionalized surface is biotinylated.

18. The method of claim 12, wherein the chemically functionalized surface is functionalized with (3-aminopropyl)triethoxysilane and biotin, and wherein the one or more detector molecules comprises streptavidin linked to the (3-aminopropyl)triethoxysilane by the biotin.

19. The method of claim 12, wherein the one or more detector molecules comprises an antibody linked to the chemically functionalized surface.

20. The method of claim 19, wherein the antibody comprises a biotinylated antibody.

21. The method of claim 20, wherein the biotinylated antibody comprises biotinylated anti-cTnI.

22. A method for making a sensor comprising:
functionalizing at least one nanobelt;
linking one or more detector molecules to the functionalized nanobelt; and
fabricating, before or after the functionalizing step, a field effect transistor comprising a substrate, a source electrode on the substrate, a drain electrode on the substrate, the functionalized nanobelt on a surface of the substrate between the source electrode and the drain electrode.
